(19) United States
(12) Patent Application Publication
(10) Pub. No.: US 2012/0071768 A1
(43) Pub. Date: Mar. 22, 2012

(54) BLOOD PRESSURE INFORMATION MEASUREMENT DEVICE

(75) Inventors: Kenichi Yamakoshi, Kanazawa-shi (JP); Masamichi Nogawa, Kanazawa-shi (JP); Shinobu Tanaka, Kanazawa-shi (JP); Takehiro Yamakoshi, Kanazawa-shi (JP); Yukiya Sawanoi, Nara-shi (JP); Kenji Fujii, Kyoto-shi (JP); Naomi Matsumura, Osaka (JP); Reiji Fujita, Kyoto-shi (JP)

(73) Assignee: OMRON HEALTHCARE CO., LTD., Kyoto-shi (JP)

(21) Appl. No.: 13/234,579

(22) Filed: Sep. 16, 2011

Related U.S. Application Data
(63) Continuation of application No. PCT/JP2010/054333, filed on Mar. 15, 2010.

(30) Foreign Application Priority Data
Mar. 19, 2009 (JP) 2009-067826

(51) Int. Cl. A61B 5/0225; A61B 5/022

(52) U.S. Cl. 600/493; 600/499

(57) ABSTRACT

A blood pressure information measurement device includes light emitting elements and light receiving elements as a plurality of pairs of first and second sensors in a cuff. While the cuff pressure is increased, combinations of the plurality of pairs of the light emitting elements and the light receiving elements are switched, and volume pulse waves are obtained from each of the combinations. After increasing the cuff pressure, for each of the volume pulse waves corresponding to the combinations, a specific volume value at a point where the amount of volume change becomes maximal is extracted. Thereafter, based on values corresponding to the maximum and minimum blood pressure and the specific volume value, the sensors for measurement are determined.

### TECHNICAL FIELD

The present invention relates to a blood pressure information measurement device and a blood pressure information measurement method. Particularly, the invention relates to a blood pressure information measurement device using an arterial volume sensor for detecting pulse waves and a blood pressure information measurement method using the device.

### BACKGROUND ART

A blood pressure information measurement device that realizes pulse wave detection by using an arterial volume sensor such as a photoelectric sensor exists. Regarding this device, it is known that when a light emitting element and a light receiving element of the photoelectric sensor are arranged on the same surface of the body, because the average depth of near infrared light getting into the body is about half of the distance between the light emitting element and the light receiving element (hereinafter, referred to as an “inter-element distance”), a semispherical region taking the inter-element distance as the diameter thereof is a measurement region.

Accordingly, when the photoelectric sensor of this configuration is used, the sensor needs to be so placed such that an artery is positioned between the light emitting element and the light receiving element. Consequently, as a procedure of placing the sensor, a procedure of searching for the position of an artery by palpation and placing the sensor to make the position become the center of the photoelectric sensor is required. This procedure can be performed at regions such as the wrist where a pulse is palpable, but at regions such as the upper arm where a pulse is impalpable, this procedure cannot be performed. Moreover, even when the position of an artery is found on the wrist by palpation, the sensor fails to be correctly arranged on the artery in some cases when the sensor is placed. This can also occur in the same manner even when sensors other than the photoelectric sensor are used.

Therefore, as a method of easily arranging sensors for detecting pulse waves on an artery, a method of arranging a plurality of sensors (a light emitting element and a light receiving element) and selecting optimal sensors among these sensors has been suggested (Patent Documents 1 to 3, for example).

In this suggestion, a method of detecting optimal sensors detects a sensor having a maximum amplitude of an output of a photoelectric sensor in a predetermined state, a sensor having a maximum S/N (Signal to Noise) ratio, and a sensor having the highest autocorrelation as optimal sensors.
Patent Document 1: Japanese Unexamined Patent Publication No. 01-249036
Patent Document 2: Japanese Unexamined Patent Publication No. 07-299043
Patent Document 3: Japanese Unexamined Patent Publication No. 2006-271896

### SUMMARY OF INVENTION

However, when such a method is used particularly for the wrist or the like, during a process of compressing the wrist with a cuff, the sensor slants by being affected by tendons or bones, or the artery sinks beneath the tendon or the like in some cases, such that the change in arterial volume accompanied with the compression of the cuff fails to be accurately detected in some cases.

Therefore, one or more embodiments of the present invention provide a blood pressure information measurement device that enables any subject to easily detect volume pulse waves with a high accuracy and to provide a measuring method using this device.

A blood pressure information measurement device according to one or more embodiments of the invention is a blood pressure information measurement device for measuring blood pressure information by detecting arterial volume, and the device includes a cuff to be wound around a predetermined measurement site; adjustment portions for adjusting internal pressure of the cuff by increasing or reducing the pressure; pressure detection portions for detecting a cuff pressure indicating the internal pressure of the cuff; and a volume detection portion which is arranged in a predetermined position of the cuff and used for detecting arterial volume signals showing arterial volume, wherein the volume detection portion further includes a plurality of pairs of first and second sensors, driving control portions for gradually increasing or reducing the cuff pressure until the cuff pressure reaches a specific pressure value by controlling driving of the adjustment portions, and detection process portions for performing a process of detecting a pair of sensors for measurement among the plurality of pairs of the first and second sensors. The process performed by the detection process portions includes a step of obtaining volume pulse waves by obtaining arterial volume signals from the volume detection portion for each combination of the plurality of pairs of the first and second sensors in parallel with the control performed by the driving control portions; a step of extracting a specific volume value in a point where the amount of volume change becomes maximal for each volume pulse wave corresponding to the combination, when the cuff pressure has reached a specific pressure value by the driving control portions; and a step of determining a combination of the first and second sensors in which the relationship between the specific volume value and a first volume value at a point of time when the cuff pressure becomes a value corresponding to a maximum arterial blood pressure satisfies a predetermined first relationship, and/or the relationship between the specific volume value and a second volume value at a point of time when the cuff pressure becomes a value corresponding to a minimum arterial blood pressure satisfies a predetermined second relationship, as sensors for measurement. The blood pressure information measurement device further includes a blood pressure information calculation portion for measuring the blood pressure information by using the sensors for measurement determined by a determination portion.

According to one or more embodiments of the present invention, the predetermined first relationship shows a relationship in which the specific volume value is equal to or smaller than the first volume value and a difference between the specific volume value and the first volume value is within a predetermined value.

According to one or more embodiments of the present invention, the predetermined second relationship shows a relationship in which the specific volume value is equal to or larger than the second volume value and a difference between the specific volume value and the second volume value is within a predetermined value.

According to one or more embodiments of the present invention, the blood pressure information measurement device further includes a switching portion for switching the combinations of the first and second sensors, and in the step of obtaining the volume pulse waves, the volume pulse wave for each combination is obtained while the switching portion switches the combinations of the first and the second sensors.

According to one or more embodiments of the present invention, in the step of obtaining the volume pulse waves, the combinations of the first and the second sensors are sequentially switched at the same cuff pressure.

According to one or more embodiments of the present invention, the blood pressure information measurement device further includes a storage portion for storing values of the arterial volume signals obtained in the step of obtaining the volume pulse wave by associating the values with the cuff pressure for each of the combinations, and in the step of determining the sensors for measurement, a first envelope tangent to points where the arterial volume becomes maximal for each of pulse wave components included in the volume pulse wave and a second envelope connecting points where the arterial volume becomes minimal are extracted for each of the volume pulse wave, a value of the first envelope which corresponds to a point of time when the cuff pressure of a value corresponding to the maximum blood pressure is detected is determined as a first volume value, and a value of the second envelope which corresponds to a point of time when the cuff pressure of a value corresponding to the minimum blood pressure is detected is determined as a second volume value.

Alternatively, according to one or more embodiments of the present invention, the blood pressure information measurement device further includes a storage portion for storing the values of the arterial volume signals obtained in the step of obtaining the volume pulse waves by associating the values with the cuff pressure for each of the combinations. According to one or more embodiments of the present invention, the value of the arterial volume signal increases according to the increase in the cuff pressure, and in the step of determining the sensors for measurement, average movement values of the arterial volume signals from the detection starting point are calculated, an average movement value which corresponds to a point of time when the cuff pressure of a value corresponding to the maximum blood pressure is detected is determined as the first volume value, and an average movement value which corresponds to a point of time when the cuff pressure of a value corresponding to the minimum blood pressure is detected is determined as the second volume value.

Alternatively, according to one or more embodiments of the present invention, the blood pressure information measurement device further includes a storage portion for storing the values of the arterial volume signals obtained in the step of obtaining the volume pulse waves by associating the values with the cuff pressure for each of the combinations. According to one or more embodiments of the present invention, the value of the arterial volume signal increases according to the increase in the cuff pressure, and in the step of determining the sensors for measurement, an average of the volume pulse wave for one pulse which corresponds to a point of time when the cuff pressure of a value corresponding to the maximum blood pressure is detected is determined as the first volume value, and an average of the volume pulse wave for one pulse which corresponds to a point of time when the cuff pressure of a value corresponding to the minimum blood pressure is detected is determined as the second volume value.

According to one or more embodiments of the present invention, the blood pressure information measurement device further includes a blood pressure estimation portion for estimating a maximum arterial blood pressure value and a minimum arterial blood pressure value in parallel with the control performed by the driving control portions, and each of the value corresponding to the maximum blood pressure and the value corresponding to the minimum blood pressure indicates the maximum blood pressure value and the minimum blood pressure value which are estimated by the blood pressure estimation portion.

According to one or more embodiments of the present invention, the volume detection portion is a photoelectric sensor, and the first and second sensors are a light emitting element and a light receiving element respectively.

Alternatively, according to one or more embodiments of the present invention, the volume detection portion is an impedance sensor, and the first and second sensors are a current applying electrode and a voltage measuring electrode respectively.

According to one or more embodiments of the present invention, the measuring method using a blood pressure information measurement device is a method of measuring the blood pressure information by detecting arterial volume by using a blood pressure information measurement device including a cuff for being wound around a predetermined measurement site and a plurality of pairs of first and second sensors in a predetermined position of the cuff. The method includes a step of gradually increasing or reducing cuff pressure until the cuff pressure reaches a specific pressure value; a step of obtaining volume pulse waves by obtaining arterial volume signals showing arterial volume from the sensor pairs for each of the combinations of the plurality of pairs of the first and second sensors in parallel with the increase or reduction of the cuff pressure; a step of extracting a specific volume value at a point where the amount of volume change becomes maximal for each volume pulse wave corresponding to the combination, when the cuff pressure has reached a specific pressure value; a step of determining a combination of the first and second sensors in which the relationship between the specific volume value and a first volume value at a point of time when the cuff pressure becomes a value corresponding to a maximum arterial blood pressure satisfies a predetermined first relationship, and/or the relationship between the specific volume value and a second volume value at a point of time when the cuff pressure becomes a value corresponding to a minimum arterial blood pressure satisfies a predetermined second relationship, as sensors for measurement; and a step of measuring blood pressure information by using the determined sensors for measurement.

According to one or more embodiments of the present invention, in the process of gradually increasing or reducing the cuff pressure, the volume pulse wave is obtained for each of the combinations of the plurality of pairs of the first and second sensors, whereby a sensor pair which is optimal as the sensors for measurement is determined based on the relationship between the specific volume value and the first volume value and/or the second volume value in the obtained volume pulse waves. Accordingly, it is possible to easily align the position of the sensors on the artery.

Moreover, because the sensors for measurement are determined using the volume pulse waves obtained in the process of gradually increasing or reducing the cuff pressure, it is possible to accurately detect even the change in arterial volume accompanied with the compression of the cuff in measuring the blood pressure information.

### BRIEF DESCRIPTION OF THE DRAWINGS

FIG. 1 is a perspective view of the exterior of a blood pressure information measurement device according to one or more embodiments of the invention.

FIG. 2 is a block diagram illustrating a hardware configuration of the blood pressure information measurement device according to one or more embodiments of the invention.

FIG. 3A is a view illustrating an arrangement example of a plurality of pairs of a light emitting element and a light receiving element.

FIG. 3B is a view illustrating an arrangement example of the plurality of pairs of the light emitting element and the light receiving element.

FIG. 3C is a view illustrating an arrangement example of the plurality of pairs of the light emitting element and the light receiving element.

FIG. 3D is a view illustrating an arrangement example of the plurality of pairs of the light emitting element and the light receiving element.

FIG. 4 is a view illustrating an example of specific intervals for arranging the plurality of pairs of the light emitting element and the light receiving element.

FIG. 5 is a functional block diagram illustrating a functional configuration of the blood pressure information measurement device according to one or more embodiments of the invention.

FIG. 6 is a view illustrating an example of a data structure of information on volume pulse waves stored in a memory portion.

FIG. 7 is a view illustrating a typical example of the volume pulse waves obtained while an artery is compressed, and the ideal relationship between a specific volume value and other volume values.

FIG. 8 is a view illustrating dynamical characteristics of the artery.

FIG. 9 is a view illustrating the relationship between presumable specific volume values and other volume values.

FIG. 10 is a view illustrating a typical example of the volume pulse waves detected by a combination of sensors which is arranged in an abnormal position.

FIG. 11 is a flowchart illustrating a blood pressure measuring process performed by the blood pressure information measurement device according to one or more embodiments of the invention.

FIG. 12 is a view illustrating an example of a screen displayed in step S32 in FIG. 11.

FIG. 13 is a view illustrating an example of a data structure of measured data.

FIG. 14 is a view illustrating a line connecting average movement values of arterial volume signals from a measurement starting point.

FIG. 15 is a view illustrating a line connecting averages of the arterial volume signal per pulse.

FIG. 16 is a view illustrating an arrangement example of a plurality of pairs of a current electrode and a voltage electrode.

FIG. 17A is a conceptual view illustrating an arrangement example of a photoelectric sensor in which a light emitting element and a light receiving element are arranged separately in a normal arrangement state in a general blood pressure information measurement device.

FIG. 17B is a conceptual view illustrating an arrangement example of the photoelectric sensor in which the light emitting element and the light receiving element are arranged separately in an abnormal arrangement state in the general blood pressure information measurement device.

FIG. 18A is a conceptual view illustrating an arrangement example of the photoelectric sensor in which the light emitting element and the light receiving element are arranged integrally in a normal arrangement state in the general blood pressure information measurement device.

FIG. 18B is a conceptual view illustrating an arrangement example of the photoelectric sensor in which the light emitting element and the light receiving element are arranged integrally in an abnormal arrangement state in the general blood pressure information measurement device.

FIG. 18C is a conceptual view illustrating an arrangement example of the photoelectric sensor in which the light emitting element and the light receiving element are arranged integrally in an abnormal arrangement state in the general blood pressure information measurement device.

### DETAILED DESCRIPTION OF INVENTION

Embodiments of the invention will be described in detail with reference to the drawings. In addition, in the drawings, portions identical or corresponding to each other are marked with the same reference numerals, whereby the description thereof will not be repeated.

The blood pressure information measurement device according to one or more embodiments of the present invention includes both an arterial volume sensor for detecting arterial volume signals showing the arterial volume and a pressure sensor for detecting the internal cuff pressure. Accordingly, the arterial volume sensor will be described as a photoelectric sensor.

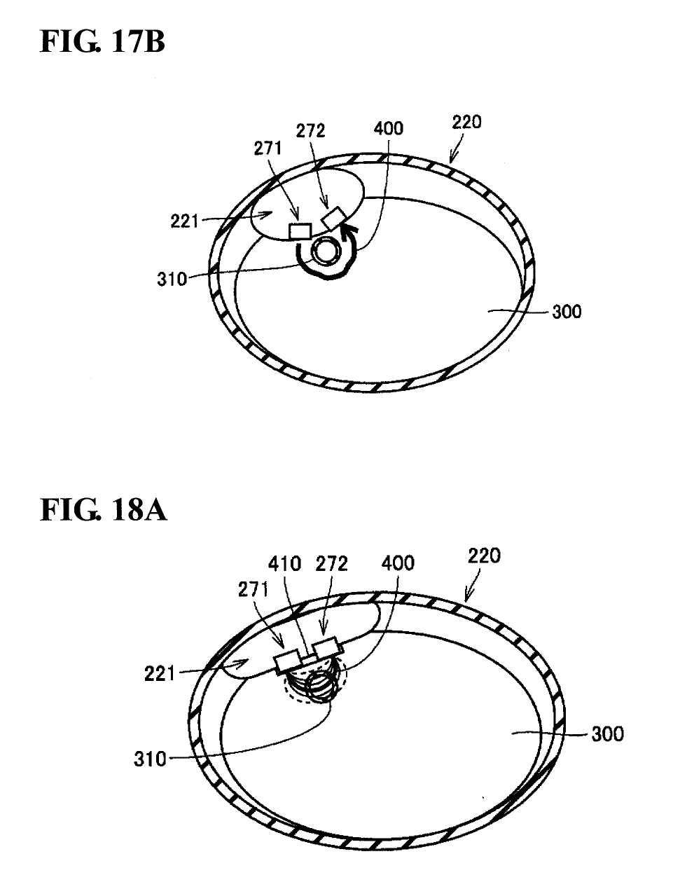

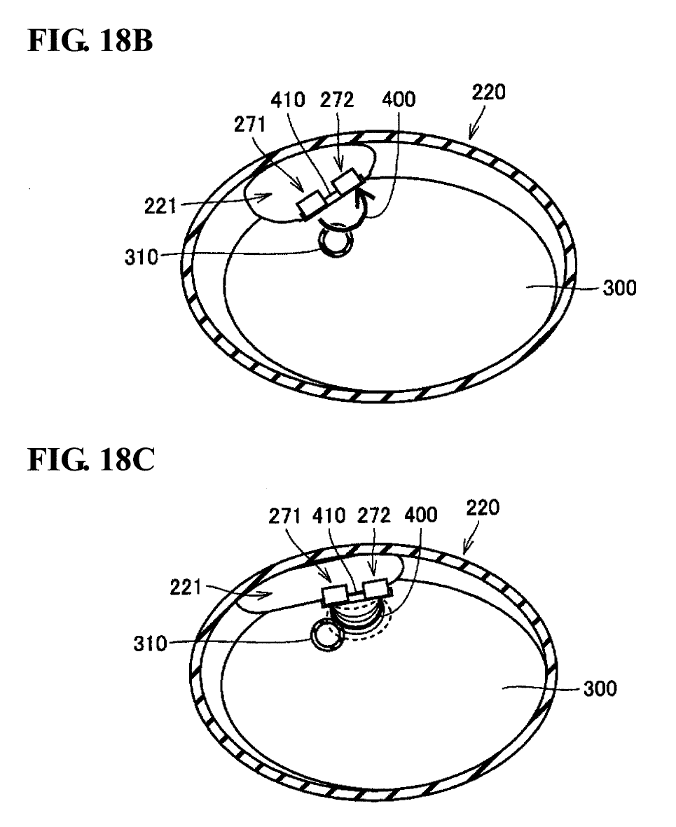

First, an arrangement example of the photoelectric sensor in a general blood pressure information measurement device will be described by using FIGS. 17A and 17B and FIGS. 18A to 18C. In the general blood pressure information measurement device, only a pair of photoelectric sensors is arranged in a cuff.

FIGS. 17A and 17B are conceptual views illustrating an arrangement example of a photoelectric sensor in which a light emitting element 271 and a light receiving element 272 are separately arranged in the general blood pressure information measurement device. FIGS. 18A to 18C are conceptual views illustrating an arrangement example of the photoelectric sensor in which the light emitting element 271 and the light receiving element 272 are integrally arranged by a substrate 410 in the general blood pressure information measurement device. The photoelectric sensor may be arranged in the outer surface of a cuff 220 contacting a body 300 or may be arranged in the inside of the surface (hereinafter, referred to as an “inner surface”) of the cuff 220 contacting the body 300.

In FIGS. 17A and 18A, even while an airbag 221 of the cuff 220 expands, the light emitting element 271 and the light receiving element 272 are arranged on the surface right above an artery 310 in the body 300 in a normal state. That is, because the artery 310 is included in the measurement region of the light emitting element 271 and the light receiving element 272, an optical axis 400 can penetrate the artery 310. Therefore, in this state, volume change of the artery 310 is satisfactorily detected by the light emitting element 271 and the light receiving element 272.

Contrary to this, as shown in FIG. 17B, when the light emitting element 271 and the light receiving element 272 are separately arranged, each of a light emitting surface and a light receiving surface of the elements 271 and 272 slant in different directions respectively due to the shape change of the airbag 221 in some cases. Moreover, as shown in FIG. 18B, even if the elements 271 and 272 are fixed onto the substrate 410, the elements slant along with the substrate 410 in some cases.

As shown in FIG. 18C, if the center of the elements 271 and 272 is arranged at a position deviating from the artery 310 at a point of time when the cuff 220 is applied on the body 300, the volume change of the artery 310 fails to be satisfactorily detected.

Contrary to this, the blood pressure information measurement device according to one or more embodiments of the present invention includes a plurality of pairs of photoelectric sensors (the light emitting element and the light receiving element), so it is possible to detect (determine) optimal sensors for detecting the volume pulse wave among these sensors. Accordingly, regardless of a direct attaching type of device and a built-in type of device, it is possible to measure the volume pulse wave with a high accuracy without burdening a subject and a measurement assistant such as a physician.

As used herein, “blood pressure information” refers to information showing characteristics of the circulatory system. The blood pressure information includes at least the pulse wave, and in addition to the pulse wave, the information further includes indices that can be calculated from the pulse wave, such as a maximum blood pressure, minimum blood pressure, mean blood pressure value, pulse, AI (Augmentation Index) value, and the like.

A pulse wave which is one of the blood pressure information is divided into a pressure pulse wave and a volume pulse wave depending on how the pulse wave is treated. The pressure pulse wave is obtained by treating the pulse wave as the cuff pressure change according to the cuff volume change, by means of converting the intravascular volume change according to heart beats into the cuff volume change. The pressure pulse wave can be obtained based on the output of the pressure sensor. The volume pulse wave is obtained by treating the pulse wave as the intravascular volume change according to heart beats, and can be obtained based on the output of the arterial volume sensor. Herein, because the intravascular volume change is a phenomenon caused by the intravascular pressure change, the pressure pulse wave and the volume pulse wave can be mentioned as indices having almost the same meaning medically. Moreover, the intravascular volume change can be treated as the change in blood tissue amount in blood vessels.

The term blood pressure information measurement device used in the present specification refers to the entire device having at least a function of obtaining the pulse wave, and more specifically, the term refers to a device that obtains the volume pulse wave by detecting the change in blood tissue amount through an optical method. In this sense, the blood pressure information measurement device is not limited to a device outputting the obtained volume pulse wave as a measurement result as it is, and also includes a device outputting only the specific indices described above which are calculated or measured based on the obtained volume pulse wave as the measurement result, and a device outputting both the volume pulse wave and the specific indices as the measurement result.

In the embodiment, the blood pressure information measurement device refers to a device which detects the volume pulse wave and measures the maximum blood pressure value and the minimum blood pressure value based on the detected volume pulse wave.

<Exterior and Configuration>

(Exterior)

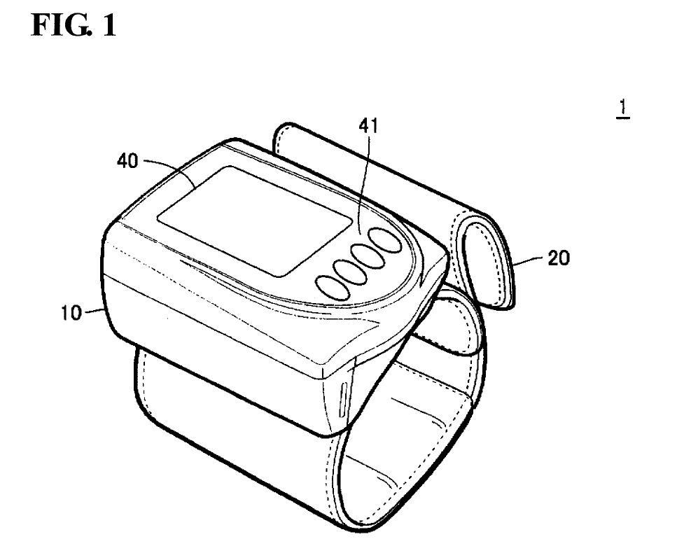

FIG. 1 is a perspective view of the exterior of a blood pressure information measurement device 1 (hereinafter, referred to as a “sphygmomanometer”) according to one or more embodiments of the invention.

As shown in FIG. 1, the sphygmomanometer 1 includes a body portion 10, and a cuff 20 that can be wound around the wrist of a subject. The body portion 10 is installed in the cuff 20. On the surface of the body portion 10, for example, a display portion 40 configured with a liquid crystal or the like and an operation portion 41 for receiving instructions from a user (typically, the subject) are arranged. The operation portion 41 includes a plurality of switches, for example.

According to one or more embodiments of the present invention, the cuff 20 is described as being applied to the wrist of the subject. However, the site (measurement site) to which the cuff 20 is applied is not limited to the wrist, and for example, the site may also include the upper arm.

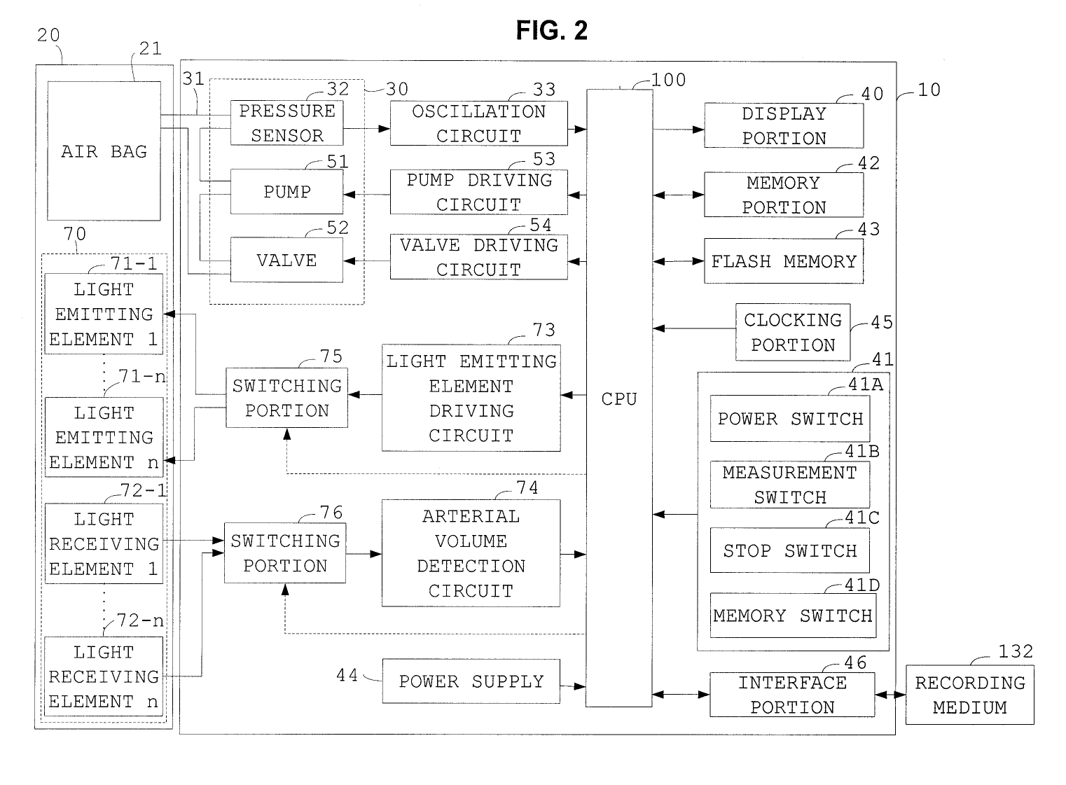

For the sphygmomanometer 1 according to one or more embodiments of the present invention, an embodiment in which the body portion 10 is installed in the cuff 20 as shown in FIG. 1 will be described for example. However, an embodiment in which the body portion 10 and the cuff 20 are connected to each other by an air tube (an air tube 31 in FIG. 2), which is used for an upper arm sphygmomanometer, may be employed.

(Hardware Configuration)

FIG. 2 is a block diagram illustrating a hardware configuration of the sphygmomanometer 1 according to one or more embodiments of the invention.

As shown in FIG. 2, the cuff 20 of the sphygmomanometer 1 includes an airbag 21 and a photoelectric sensor 70. The photoelectric sensor 70 includes a plurality of light emitting elements 71-1 to 71-n and a plurality of light receiving elements 72-1 to 72-n. In the following description, the light emitting elements 71 and the light receiving elements 72 represent the elements unless any one of the plurality of light emitting elements 71-1 to 71-n and the plurality of light receiving elements 72-1 to 72-n is specifically described.

Each of the light emitting elements 71 emits light to the artery, and each of the light receiving elements 72 receives the light penetrating the artery and emitted from the light emitting elements 71.

In order to accurately detect the intra-arterial volume change, according to one or more embodiments of the present invention, near infrared light easily penetrating body tissue is used as detection light, and as the light emitting elements 71 and the light receiving elements 72, those which can emit and receive the near infrared light are suitably used, respectively. More specifically, as the detection light, which is emitted from the light emitting elements 71 and received by the light receiving elements 72, the near infrared light having a wavelength of around 940 nm is particularly suitably used. Furthermore, the detection light is not limited to the near infrared light of a wavelength of around 940 nm, and light of a wavelength of around 450 nm and light of a wavelength of around 1100 nm are also usable.

The airbag 21 is connected to an air system 30 through the air tube 31.

In addition to the display portion 40 and the operation portion 41, the body portion 10 also includes the air system 30, a CPU (Central Processing Unit) 100 for intensively controlling each portion and performing each calculation process, a memory portion 42 for storing programs causing the CPU 100 to perform a predetermined operation and various data, a non-volatile memory (for example, a flash memory) 43 for storing the measured blood pressure, a power supply 44 for supplying power to the CPU 100, a clocking portion 45 performing a clocking operation, and an interface portion 46 for reading or writing programs or data from or on a recording medium 132 that can be attached or detached.

The operation portion 41 includes a power switch 41A registering the input of instructions for turning the power ON or OFF, a measurement switch 41B for registering an instruction for starting the measurement, a stop switch 41C for registering an instruction for stopping the measurement, and a memory switch 41D for registering an instruction for reading information such as blood pressure recorded in the flash memory 43.

The air system 30 includes a pressure sensor 32 for detecting pressure (cuff pressure) in the airbag 21, a pump 51 for supplying air to the airbag 21 to increase the cuff pressure, and a valve 52 opened or closed for discharging or sealing the air in the airbag 21.

The body portion 10 includes a light emitting element driving circuit 73, an arterial volume detection circuit 74, and switching portions 75 and 76, and further includes an oscillation circuit 33, a pump driving circuit 53, and a valve driving circuit 54, in relation to the air system 30.

The switching portion 75 is connected to all of the light emitting elements 71-1 to 71-n and to the light emitting element driving circuit 73. The switching portion 75 selects one of the light emitting elements 71 in response to a command signal from the CPU 100. As a result, current from the light emitting element driving circuit 73 is selectively output to the light emitting elements 71.

The light emitting element driving circuit 73 causes the light emitting elements 71 to emit light based on a control signal of the CPU 100. By applying a predetermined amount of current to the light emitting elements 71, the light emitting element driving circuit 73 causes the light emitting elements 71 to emit light.

The switching portion 76 is connected to all of the light receiving elements 72-1 to 72-n and to the arterial volume detection circuit 74. The switching portion 76 selects one of the light receiving elements 72 in response to a command signal from the CPU 100. As a result, output signals from the light receiving elements 72 are selectively output to the arterial volume detection circuit 74.

The arterial volume detection circuit 74 detects the arterial volume by converting an output from one light receiving element 72 obtained through the switching portion 76 into a voltage value. The arterial volume detection circuit 74 includes processing circuits such as an analog filtering circuit, an amplifier circuit, and an A/D (Analog/Digital) converting circuit, for example, and outputs signals input as analog values as voltage signals by digitalizing the signals.

Functions of the switching portions 75 and 76 are configured by switches, for example. In addition, the switching portions 75 and 76 may be included in the light emitting element driving circuit 73 and the arterial volume detection circuit 74, respectively.

The pressure sensor 32 is a capacitance type of pressure sensor, and the capacity value thereof varies with the cuff pressure. The oscillation circuit 33 outputs signals of oscillation frequency corresponding to the capacity value of the pressure sensor 32 to the CPU 100. The CPU 100 detects pressure by converting the signal obtained from the oscillation circuit 33 into pressure. The pump driving circuit 53 controls driving of the pump 51 based on the control signal provided from the CPU 100. The valve driving circuit 54 controls the valve 52 to be opened or closed based on the control signal provided from the CPU 100.

Although the cuff 20 includes the airbag 21, the fluid supplied to the cuff 20 is not limited to air, and the fluid may be liquid or gel, for example. Alternatively, the substance supplied to the cuff 20 is not limited to fluid, and may be uniform fine particles such as microbeads.

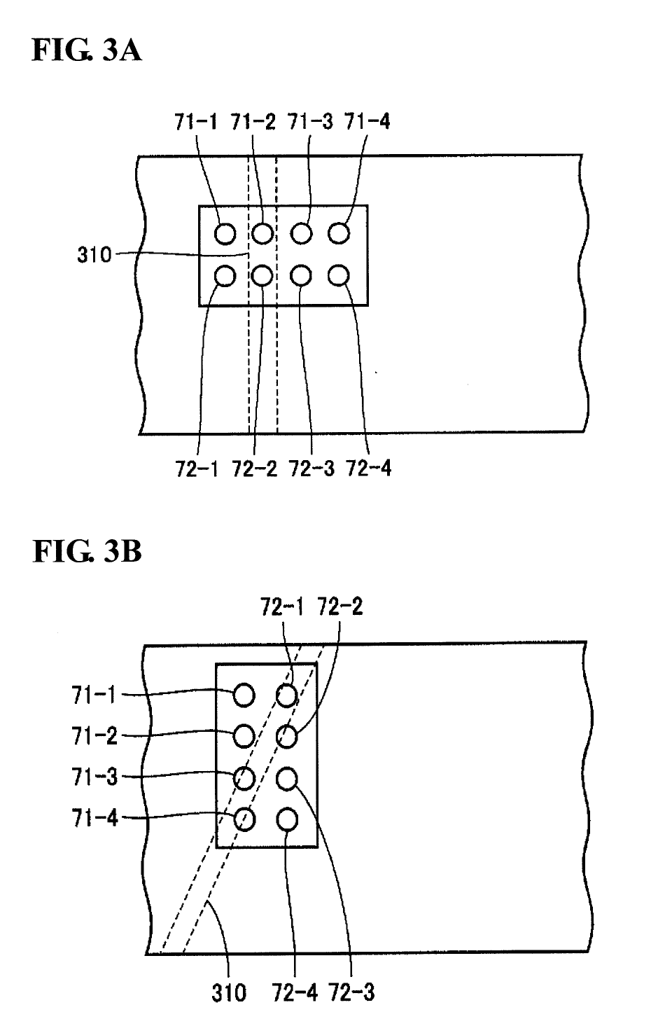

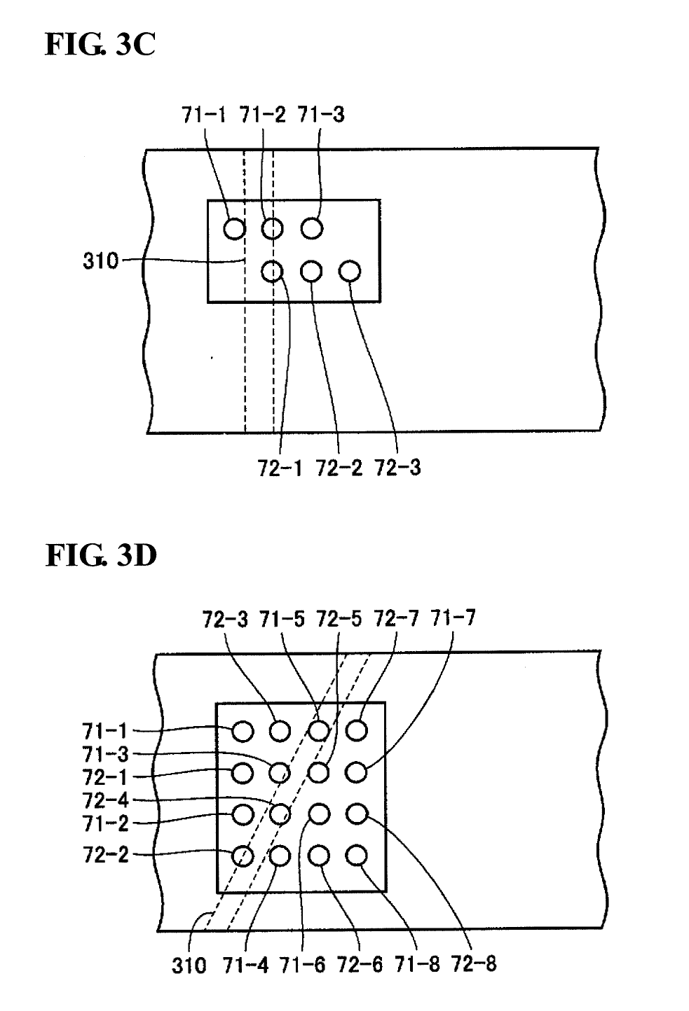

Herein, an arrangement example of the plurality of light emitting elements 71-1 to 71-n and the plurality of light receiving elements 72-1 to 72-n will be described using FIGS. 3A to 3D. In FIGS. 3A to 3D, a predetermined portion in the surface of the cuff 20 contacting the body (hereinafter, referred to as a “contact surface”) is illustrated. The horizontal direction in the drawing indicates the longitudinal direction of the cuff 20.

As shown in FIG. 3A, in a first arrangement example, 4 pairs of light emitting elements 71-1 to 71-4 and light receiving elements 72-1 to 72-4 are arranged in a matrix shape. In a first row (longitudinal direction of the cuff 20) of the contact surface of the cuff 20, 4 light emitting elements 71-1 to 71-4 are arranged at predetermined intervals, and in a second row, 4 light receiving elements 72-1 to 72-4 are arranged at the same intervals.

According to the first arrangement example, when the cuff 20 is applied, as long as the vertical direction (axial direction of the wrist) of the photoelectric sensor 70 is aligned so as to cover the position of the artery 310 to be measured, it is possible to satisfactorily measure the volume change of the artery 310 even if the horizontal direction (circumferential direction of the wrist) of the sensor deviates a little.

As shown in FIG. 3B, in a second arrangement example, 4 pairs of light emitting elements 71-1 to 71-4 and light receiving elements 72-1 to 72-4 are also arranged in a matrix shape. In the second arrangement example, 4 light emitting elements 71-1 to 71-4 are arranged in a first column at predetermined intervals, and 4 light receiving elements 72-1 to 72-4 are arranged in a second column at the same intervals.

According to the second arrangement example, when the cuff 20 is applied, as long as the horizontal direction of the photoelectric sensor 70 is aligned in the position of the artery 310 to be measured, it is possible to satisfactorily measure the volume change of the artery 310 even if the sensor deviates a little in the vertical direction.

As shown in FIG. 3C, a third arrangement example shows a modified example of the first arrangement example. In this example, 3 light emitting elements 71-1 to 71-3 are arranged in the first row at predetermined intervals, and 3 light receiving elements 72-1 to 72-3 are arranged in the second row at the same intervals. A pair of the light emitting element 71-2 and the light receiving element 72-1 and a pair of the light emitting element 71-3 and the light receiving element 72-2 are arranged in the same column, respectively.

As shown in FIG. 3D, a fourth arrangement example shows an example including the first to third arrangement examples. In the fourth arrangement example, light emitting elements 71 and light receiving elements 72 are alternatively arranged in each of the rows and columns.

According to the fourth modified example, even if the cuff 20 deviates in any of the horizontal, vertical, and oblique directions, it is possible to satisfactorily measure the volume change of the artery 310.

According to one or more embodiments of the present invention, for example, the first arrangement example is employed because it is generally considered that horizontal positioning is difficult. In addition, according to one or more embodiments of the present invention, the plurality of photoelectric sensors 70 may be arranged in the outer surface (the surface contacting the body) or arranged in (built in) the inner surface of the cuff 20.

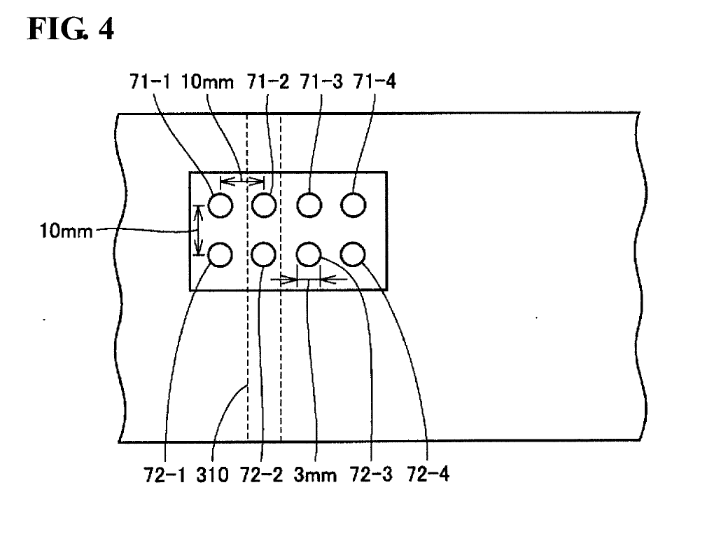

FIG. 4 is a view illustrating a specific example of intervals for arranging the light emitting elements 71-1 to 71-4 and the light receiving elements 72-1 to 72-4.

As shown in FIG. 4, each element may be a circular element with a diameter of about 3 mm, and the elements may be arranged at an interval of about 10 mm as the inter-element distance (distance between midpoints of neighboring elements). The size, shape, inter-element distance, and the number of the elements shown in FIG. 4 are just examples, and according to one or more embodiments of the present invention, the distance between one end and the other end of the elements lined up in the horizontal direction are arranged so as to cover about a fourth of the wrist of a typical male adult. Therefore, when the element with the size described above is used, according to one or more embodiments of the present invention, 4 to 5 elements are arranged in the horizontal direction of the cuff 20.

(Functional Configuration)

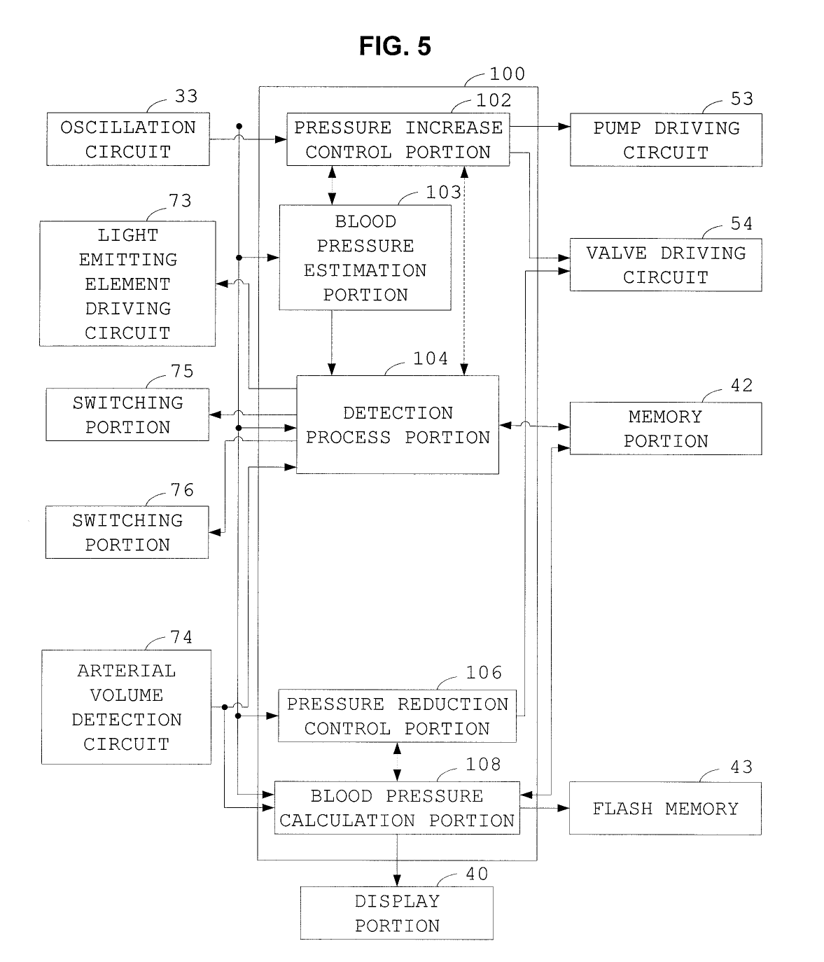

FIG. 5 is a functional block diagram illustrating a functional configuration of the sphygmomanometer 1 according to one or more embodiments of the invention. To simplify the description, FIG. 5 illustrates only peripheral hardware that directly exchanges signals with each portion of the CPU 100.

As shown in FIG. 5, the CPU 100 includes a pressure increase control portion 102, a blood pressure estimation portion 103, a detection process portion 104, a pressure reduction control portion 106, and a blood pressure calculation portion 108, as a functional configuration.

The pressure increase control portion 102 controls the pump driving circuit 53 and the valve driving circuit 54 so as to control the internal pressure of the cuff 20 to be increased to a specific pressure value. The “specific pressure value” may be a predetermined pressure (for example, 160 mmHg) or may be a value higher than a maximum blood pressure (a value corresponding to a maximum blood pressure) estimated by the blood pressure estimation portion 103 described later by a predetermined value (for example, 40 mmHg). Alternatively, while a user (subject) keeps pressing the measurement switch 41B, the pressure may be increased continuously, and in this case, the specific pressure value may be the cuff pressure at a point of time when the measurement switch 41B has been released from pressing. The specific pressure value according to one or more embodiments of the present invention refers to a predetermined pressure.

While the pressure is increased, the blood pressure estimation portion 103 applies a predetermined algorithm, thereby estimating (calculating) the maximum and minimum blood pressures. The estimation of the blood pressure in the process of increasing pressure has been performed hitherto, and the method thereof is not particularly limited. However, because optimal sensors for detecting the pulse wave cannot be ascertained while the pressure is increased, according to one or more embodiments of the present invention, the blood pressure is estimated according to an oscillometric method. That is, by applying a predetermined algorithm to the pulse wave amplitude superimposed on the cuff pressure, the maximum and minimum blood pressures are estimated. In addition, when the blood pressure is estimated in the process of reducing the pressure, the estimation can be performed by the same algorithm.

The reason why the maximum and minimum blood pressures are “estimated” during pressure increase is as follows. For example, when the blood pressure value is measured in the process of reducing the pressure, or when the blood pressure is continuously measured by a volume compensation method, it is necessary to increase the cuff pressure to equal to or higher than the maximum blood pressure as soon as possible. Consequently, for example, when the oscillometric method is used, a sufficient number of pulse wave amplitude that enables an accurate blood pressure measurement is not obtained during pressure increase.

In this manner, the maximum and minimum blood pressures estimated by the blood pressure estimation portion 103 are called a value corresponding to the maximum blood pressure and a value corresponding to the minimum blood pressure, respectively.

The detection process portion 104 performs a process of detecting a pair of sensors for measurement among the plurality of pairs of light emitting elements 71-1 to 71-4 and the light receiving elements 72-1 to 72-4. The sensor detection process performed by the detection process portion 104 will be described later in detail.

When the cuff pressure has reached a specific pressure value and the sensors for measurement have been detected, the pressure reduction control portion 106 controls the valve driving circuit 54, thereby controlling the cuff pressure to be reduced.

In parallel with the pressure reduction control of the pressure reduction control portion 106, the blood pressure calculation portion 108 applies a predetermined algorithm to the arterial volume signal detected by the sensors for measurement and the cuff pressure signal obtained from the oscillation circuit 33, thereby calculating the maximum and minimum blood pressures. The blood pressure calculation portion 108 may also calculate pulse rate by a well known method. Each of the calculated values is displayed on the display portion 40 and stored in a measurement result storing region of the flash memory 43 as measurement data.

The operation of each of the functional blocks included in the CPU 100 may be realized by executing software stored in the memory portion 42. Alternatively, the operation of at least one of the functional blocks may be realized by hardware.

Herein, the sensor detection process will be specifically described. In parallel with the control performed by the pressure increase control portion 102, the detection process portion 104 obtains the volume pulse wave for each combination of the light emitting elements 71-1 to 71-4 and the light receiving elements 72-1 to 72-4. That is, the detection process portion 104 obtains the arterial volume signal from the arterial volume detection circuit 74 in a time series manner for each combination of sensors. The volume pulse wave obtained for each combination of sensors is stored in the memory portion 42.

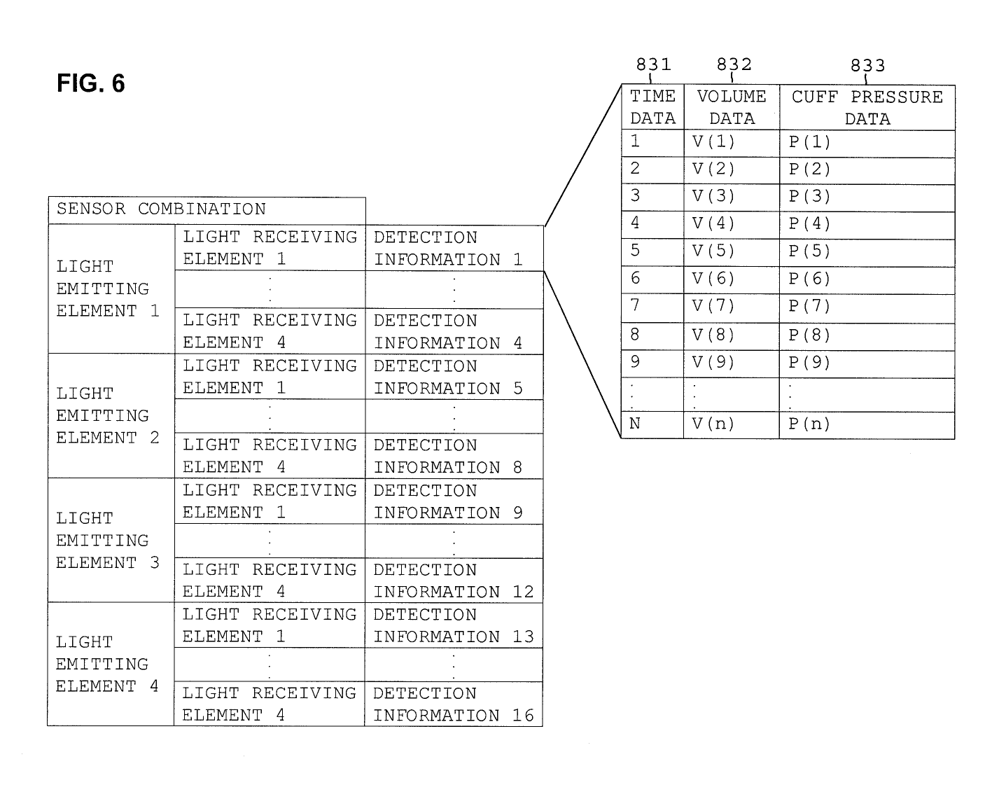

FIG. 6 is a view illustrating an example of a data structure of information on the volume pulse wave stored in the memory portion 42.

As shown in FIG. 6, the memory portion 42 stores detection information for each combination of sensors. The detection information includes time data 831 showing time, volume data (volume pulse wave data) 832 showing the value of the arterial volume signal, and cuff pressure data 833 showing the cuff pressure, and the respective data is stored by being associated with each other.

According to one or more embodiments of the present invention, the cuff pressure data is stored by being associated with the respective volume pulse wave data corresponding to the combination of sensors. However, the cuff pressure data may be stored by being related with the respective volume pulse wave data.

According to one or more embodiments of the present invention, the arterial volume signals of 16 patterns based on all combinations of 4 light emitting elements 71-1 to 71-4 and 4 light receiving elements 72-1 to 72-4 as the combination of sensors are detected. However, only the pattern based on the combination of neighboring sensors may be detected. That is, for example, the arterial volume signals of 4 patterns may be detected based on a pair of the light emitting element 71-1 and the light receiving element 72-1, a pair of the light emitting element 71-2 and the light receiving element 72-2, a pair of the light emitting element 71-3 and the light receiving element 72-3, and a pair of the light emitting element 71-4 and the light receiving element 72-4.

When the cuff pressure reaches a predetermined pressure (when the pressure increase control ends), the detection process portion 104 determines the sensors for measurement, based on the volume pulse wave data and the cuff pressure data stored in the memory portion 42.

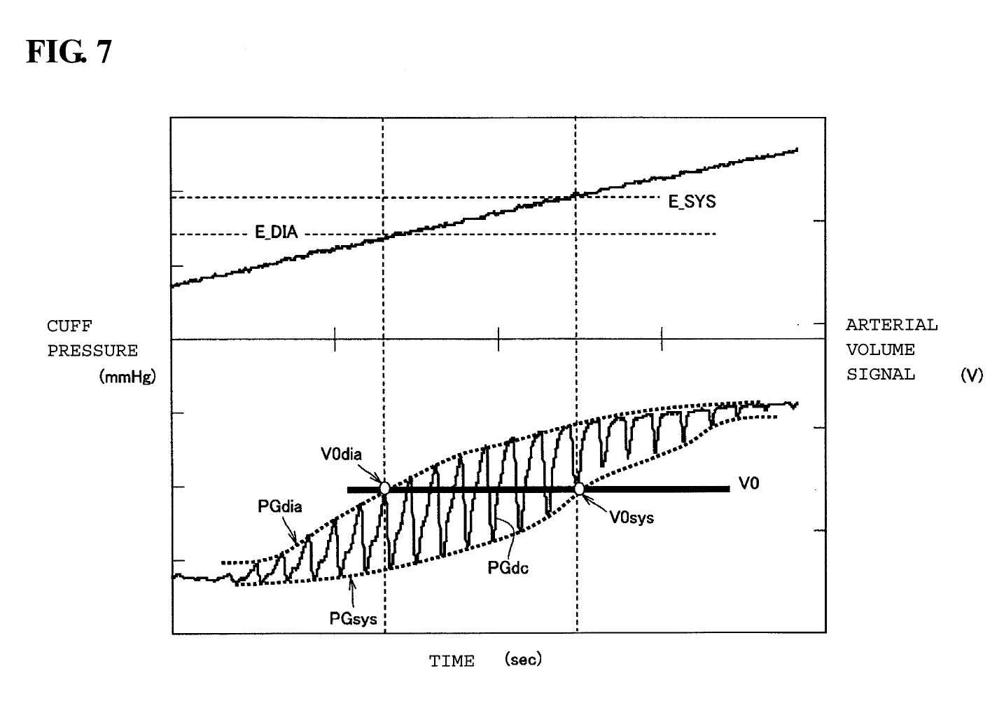

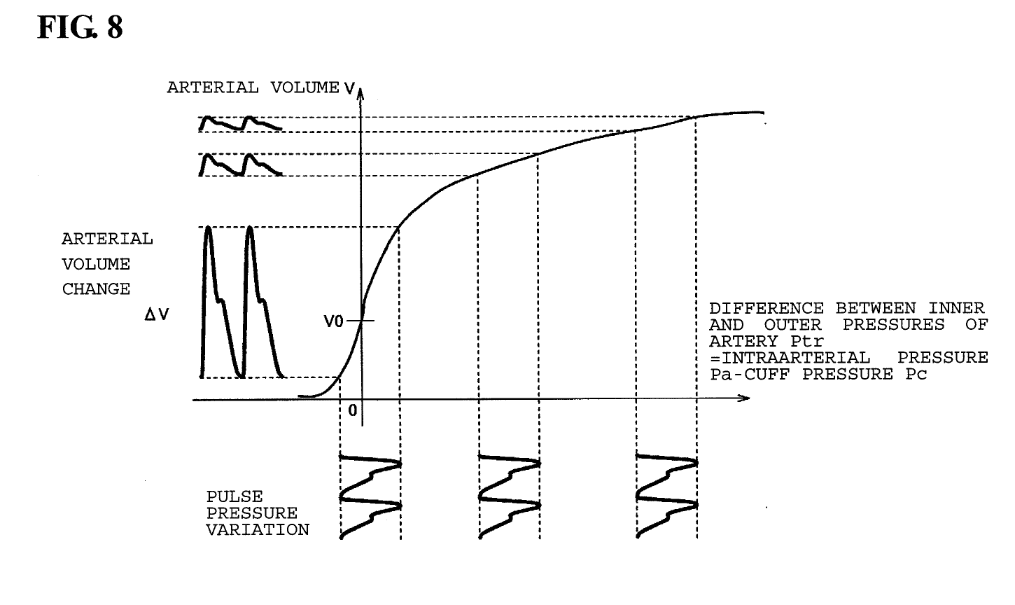

Herein, a principle for determining the sensors for measurement will be described with reference to FIGS. 7 and 8.

FIG. 7 is a view illustrating a typical example of the volume pulse wave (arterial volume signal) obtained while the artery is compressed. In the upper part of FIG. 7, signals showing the cuff pressure detected by the pressure sensor 32 are shown along a time axis clocked by the clocking portion 45. In the lower part of FIG. 7, arterial volume signals PGdc are shown along the same time axis.

As described above, in the photoelectric sensor in which the light emitting element and the light receiving element are arranged on the same surface of the body, light emitted to the body passes through a depth which is about a half of the inter-element distance, and an approximately semispherical region having the inter-element distance as a diameter thereof becomes the measurement range. When the light emitting element and the light receiving element are in an accurate position with respect to the artery to be measured (that is, a position where the inter-element distance is two times the depth of the artery, and the artery is positioned at the center between the light emitting element and the light receiving element), the value of the arterial volume signal increases as the cuff pressure increases, as shown in FIG. 7. This is because the artery is compressed by the cuff pressure, such that the arterial volume decreases, which leads to the decrease in the blood volume absorbing light.

The arterial volume varies with the pulsation of blood pressure. Therefore, within a beat of pulsation, the amount of light received by the light receiving element decreases when blood pressure is maximal (that is, when the arterial volume is large), and inversely, the amount of light received increases when blood pressure is minimal (that is, when the arterial volume is small). Accordingly, in the arterial volume signal PGdc in FIG. 7, a line connecting every maximum point per beat of pulse is shown as an arterial volume change PGdia at the time of the minimum blood pressure, and inversely, a line connecting every minimum point is shown as arterial volume change PGsys at the time of the maximum blood pressure. That is, an envelope tangent to minimum points of the arterial volume for each pulse wave component configuring the volume pulse wave is shown as the arterial volume change PGsys, and an envelope tangent to maximum points of the arterial volume for each pulse wave component is shown as arterial volume change PGdia. Each “pulse wave component” herein corresponds to the arterial volume change per beat of pulse.

Next, the dynamic characteristic of the artery is shown in FIG. 8.

FIG. 8 is a graph showing the dynamic characteristic of the artery. The graph in FIG. 8 shows the relationship between a difference between inner and outer pressures Ptr and an arterial volume V, by taking the horizontal axis as the difference between inner and outer pressures Ptr and the vertical axis as the arterial volume V. The difference between inner and outer pressures Ptr shows a difference between an intra-arterial pressure Pa and the cuff pressure Pc applied by the cuff from the outside of the body.

As shown in the graph, the dynamic characteristic of the artery shows a strong nonlinearity in general. When the difference between inner and outer pressures Ptr is 0 (equilibrium), that is, when a load is not applied to the arterial wall, compliance (an amount of volume change caused by pulsation) of the artery becomes maximal. That is, the volume change maximally complies (develops) with the pressure change.

Therefore, if a value of arterial volume signal obtained when the difference between inner and outer pressures Ptr is 0 is taken as a “specific volume value V0”, it is understood that the cuff pressure at a point of time when a maximum value of the arterial volume signal for 1 pulse becomes the specific volume value V0 is to be the minimum blood pressure, and that the cuff pressure at a point of time when a minimum value of the arterial volume signal for 1 pulse becomes the specific volume value V0 is to be the maximum blood pressure.

From the above description, the following relationship between the cuff pressure and the volume pulse wave is drawn. That is, in the combination of sensors arranged in a normal position, the PGdia value obtained when the cuff pressure is the minimum blood pressure and the PGsys value obtained when the cuff pressure is the maximum blood pressure coincide with the specific volume value V0. In this relationship, if the PGdia value obtained when the cuff pressure is a value corresponding to the minimum blood pressure E_DIA is defined as a “volume value V0dia”, and the PGsys value obtained when the cuff pressure is a value corresponding to the maximum blood pressure E_SYS is defined as a “volume value V0sys”, a combination of the light emitting element and the light receiving element obtaining the volume pulse wave in which the volume value V0dia and the volume value V0sys coincide with the specific volume value V0 can be determined as sensors for measurement.

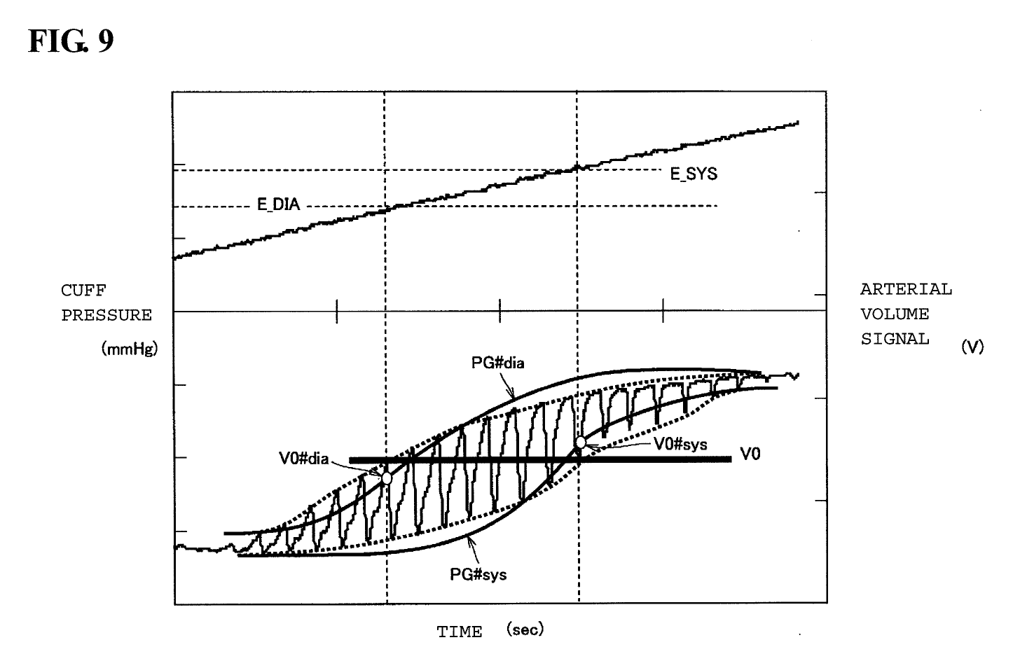

V0=V0dia=V0sys, which is a condition (predetermined relationship) making it possible to determine the sensors for measurement as described above, shows an ideal state. In practice, because the distance between the photoelectric sensor 70 and the artery is changed by the expansion of the cuff 20 resulting from pressure increase in some cases, it is assumed that each of the envelopes PGdia and PGsys would become envelopes PG#dia and PG#sys shown in FIG. 9. That is, the distance between the photoelectric sensor 70 and the artery decreases as the body is compressed by the cuff 20. Therefore, when the cuff pressure is low, both the envelopes PG#dia and PG#sys deviate below the envelopes PGdia and PGsys in FIG. 7, and inversely, when the cuff pressure is high, both the envelopes PG#dia and PG#sys deviate above the envelopes PGdia and PGsys in FIG. 7. Consequently, in the embodiment, the detection process portion 104 determines a combination of the light emitting element and the light receiving element in which the relationship of “V0dia≦V0≦V0sys” is satisfied, and a difference between V0dia and V0 and a difference between V0sys and V0 are within a predetermined value, as the sensors for measurement.

When the sensors for measurement are determined based only on the relationship between the value corresponding to maximum blood pressure E_SYS and the specific volume value V0, it is possible to determine the sensors as long as the relationship of “V0≦V0sys” is satisfied, and a difference between V0 and V0sys is within a predetermined value. In addition, when the sensors for measurement are determined based only on the relationship between the value corresponding to minimum blood pressure E_DIA and the specific volume value V0, it is possible to determine the sensors as long as the relationship of “V0≦V0dia” is satisfied, and a difference between V0 and V0dia is within a predetermined value.

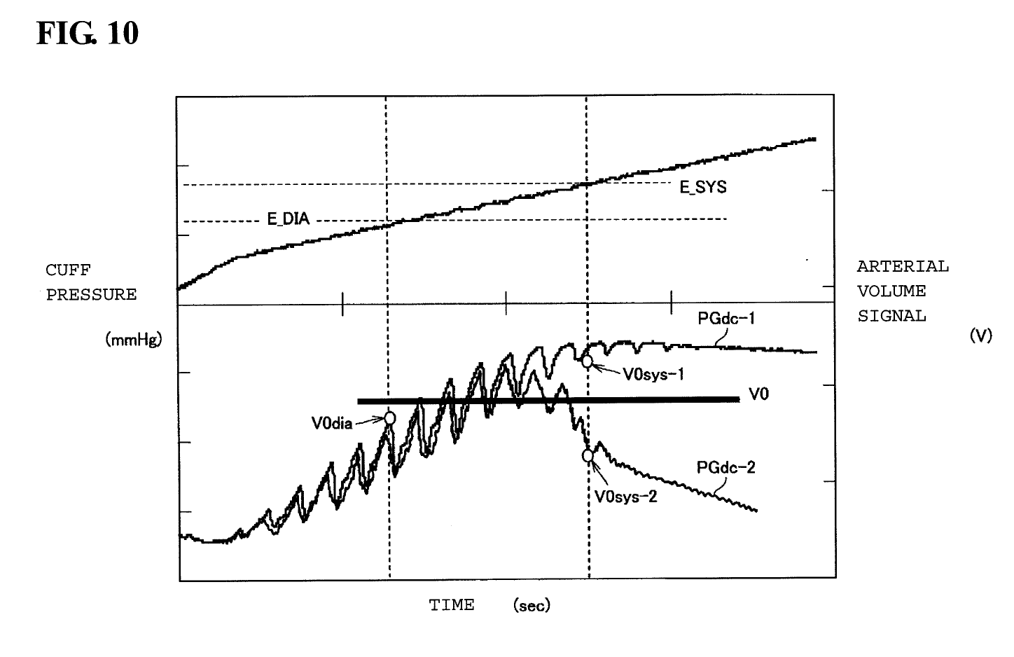

FIG. 10 is a view illustrating a typical example of the volume pulse wave detected by a combination of sensors, which is arranged in an abnormal position. The upper part and lower part of FIG. 10 show signals indicating the cuff pressure and two arterial volume signals PGdc-1 and PGdc-2 along the same time axis, respectively, similarly to FIG. 7.

As shown in FIG. 10, in the combination of the light emitting elements 71 and the light receiving elements 72, which are arranged in an abnormal position, a volume value V0sys-1 greatly exceeds the specific volume value V0 as shown by the arterial volume signal PGdc-1, or the volume value V0sys-1 is less than the specific volume value V0 as shown by the arterial volume signal PGdc-2. Therefore, for example, a sensor pair detecting the volume pulse wave such as the arterial volume signals PGdc-1 and PGdc-2 cannot be regarded as optimal sensors for blood pressure information measurement, such that these sensors cannot be determined as the sensors for measurement.

The specific volume value V0 can be calculated as an average of arterial volume signals for each pulse at points where the arterial volume change (an alternating current component of the arterial volume signal) becomes maximal. The arterial volume change signal showing the arterial volume change is extracted by filtering the signals from the arterial volume detection circuit 74, for example. Pulse wave components (arterial volume signal for one pulse) at points where the obtained value of the arterial volume change signal becomes maximal may be extracted, and the average thereof may be calculated.

According to one or more embodiments of the present invention, the minimum blood pressure and maximum blood pressure estimated during pressure increase in parallel with obtaining the volume pulse wave are taken as the value corresponding to minimum blood pressure and the value corresponding to maximum blood pressure, respectively. However, each of the corresponding values is not limited to the above values. For example, the previous measurement data stored in the flash memory 43 may also be used as the values. For example, the minimum blood pressure and the maximum blood pressure in the preceding measurement data may be used, or the averages of the minimum blood pressure and the maximum blood pressure of the measurement data of plural times of measurement may be used. Alternatively, values that the user (the subject representatively) inputs through the operation portion 41 may be used. Values based on the subject's own previous measurement data read by the attachable and detachable recording medium 132 through the interface portion 46 may also be used.

<Operation>

Next, the operation of the sphygmomanometer 1 according to one or more embodiments of the present invention will be described.

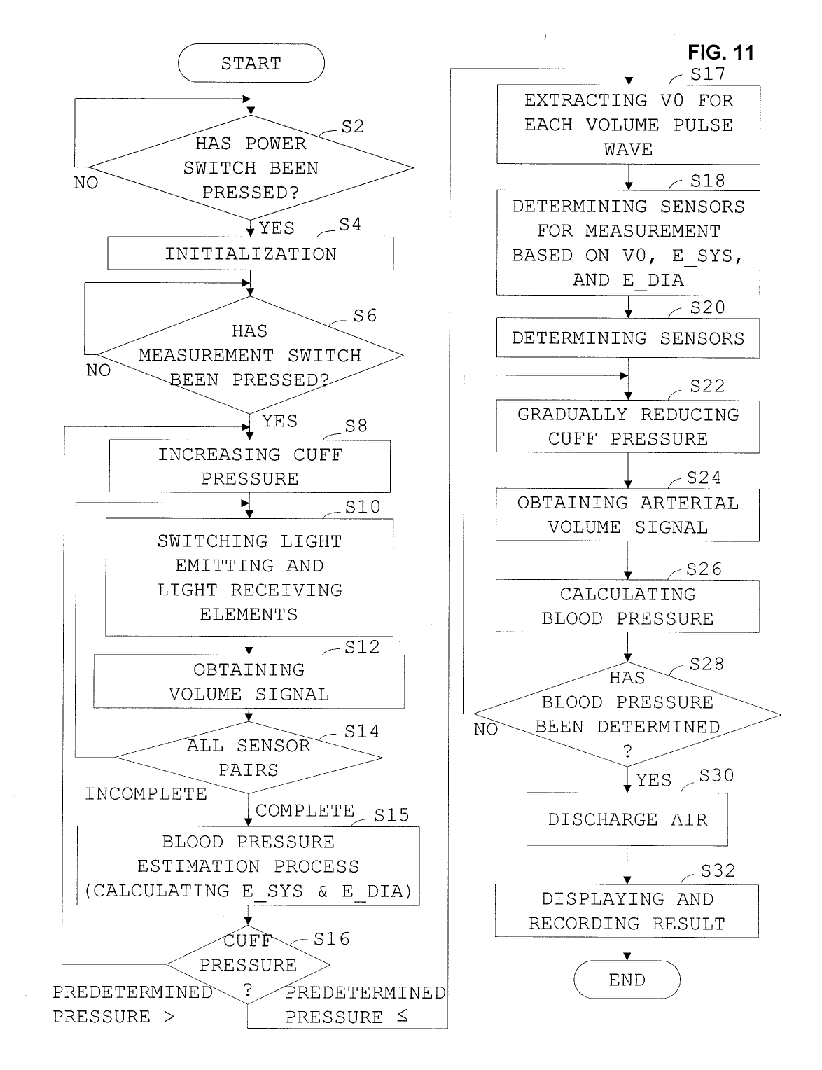

FIG. 11 is a flowchart illustrating a blood pressure information measurement process performed by the sphygmomanometer 1 according to one or more embodiments of the invention. The process shown in the flowchart in FIG. 11 is stored in advance in the memory portion 42 as a program. When the CPU 100 reads and executes this program, functions of the blood pressure information measurement process are realized.

As shown in FIG. 11, first, the CPU 100 determines whether or not the power switch 41A has been pressed (step S2). The CPU 100 waits until the power switch 41A is pressed (step S2, NO). When determining that the power switch 41A has been pressed (step S2, YES), the CPU 100 moves to step S4.

In step S4, the CPU 100 performs initialization. Specifically, the CPU 100 initializes a predetermined region of the memory portion 42, discharges air in the airbag 21, and corrects the pressure sensor 32 to be 0 mmHg.

Next, the CPU 100 determines whether or not the measurement switch 41B has been pressed (step S6). The CPU 100 waits until the measurement switch 41B is pressed (step S6, NO). When determining that the measurement switch 41B has been pressed (step S6, YES), the CPU 100 moves to step S8.

In step S8, the pressure increase control portion 102 controls the pump driving circuit 53 and the valve driving circuit 54 so as to start gradually increasing the cuff pressure. Specifically, the valve 52 is closed, and the cuff pressure is gradually increased by the pump 51.

When the pressure of the cuff 20 starts to be increased, the detection process portion 104 executes a volume pulse wave obtaining process, as a part of a sensor detection process (steps S10 to S14). Specifically, while switching the light emitting elements 71-1 to 71-4 and the light receiving elements 72-1 to 72-4 in a predetermined order by transmitting control signals to the switching portions 75 and 76 (step S10), the detection process portion 104 obtains the arterial volume signal from each combination of sensors (step S12). Until this process is completed with respect to all sensor pairs in the same pressure value (step S14, Incomplete), the processes of steps S10 and S12 are repeated. When the process is completed with respect to all sensor pairs (step S14, Complete), the process moves to step S15.

Herein, an example of the order of switching the combination of sensors will be described. First, the light emitting element 71-1 is driven. At this time, the light receiving elements 72-1 to 72-4 are driven sequentially, whereby the arterial volume signals detected by each of the light receiving elements 72 are obtained. Next, the driving of the light emitting element 71-1 is stopped, and then the light emitting element 71-2 is driven. Subsequently, the light receiving elements 72-1 to 72-4 are driven sequentially in the same manner as described above, thereby arterial volume signals detected by each of the light receiving elements 72 are obtained. Thereafter, the sensors are switched in the same manner.

According to one or more embodiments of the present invention, the arterial volume signals of all sensor pairs are obtained in the same pressure value. However, the pressure value is not necessarily limited to the same pressure value.

In step S15, the blood pressure estimation portion 103 performs a blood pressure estimation process according to the oscillometric method, for example. For example, by applying a predetermined algorithm to the pulse wave amplitude superimposed on the cuff pressure, the value corresponding to maximum blood pressure E_SYS and the value corresponding to minimum blood pressure E_DIA are calculated.

In the flowchart in FIG. 11, the blood pressure estimation process is performed in series with the sensor detection process; however, the blood pressure estimation process may be performed in parallel with the sensor detection process. When the sensors for measurement are determined by the detection process portion 104 based only on the relationship between the specific volume value V0 and the volume value V0sys, only the value corresponding to maximum blood pressure E_SYS may be calculated.

If the arterial volume signals are completely obtained from all sensor pairs in the same pressure value, the pressure increase control portion 102 determines whether or not the cuff pressure has reached a predetermined pressure (step S16). When it is determined that the cuff pressure has not reached a predetermined pressure (step S16, “predetermined pressure>”), the process returns to step S8, and the process described above is repeated. As a result, the volume pulse wave data of 16 patterns corresponding to all combinations of sensors is temporarily stored in the memory portion 42 by being associated with the cuff pressure data.

When the cuff pressure has reached the predetermined value (step S16, “predetermined value.”), the pump 51 is stopped to stop the increase of pressure, and an optimal sensor determination process is executed. That is, first, the specific volume value V0 is extracted for each volume pulse wave of 16 patterns stored in the memory portion 42 (step S17). Thereafter, based on the value corresponding to maximum blood pressure E_SYS and the value corresponding to minimum blood pressure E_DIA, which are estimated in the blood pressure estimation process in step S15, and on the specific volume value V0 extracted in step S17, the sensors for measurement are determined (step S18). The detail of the method of determining the sensors for measurement was described as above, so the description thereof will not be repeated herein.

According to one or more embodiments of the present invention, after the specific volume value V0 is extracted with respect to each pulse wave, whether a predetermined relationship is satisfied with respect to each pulse wave is determined. However, the extraction of the specific volume value V0 and the determination on the predetermined relationship may be performed in series for each pulse wave.

When the sensors for measurement are determined by the sensor determination process, the CPU 100 transmits control signals to the switching portions 75 and 76, thereby determining a pair of the light emitting element 71 and the light receiving element 72 determined as the sensors for measurement as a sensor pair (step S20).

When a sensor pair is determined as the sensors for measurement, the pressure reduction control portion 106 controls the degree of opening of the valve 52, thereby gradually reducing the cuff pressure (step S22). In addition, in parallel with the pressure reduction control with respect to the cuff 20, the arterial volume signal detected by the sensors for measurement is obtained (step S24). Subsequently, based on the obtained arterial volume signal and the cuff pressure at this time, the blood pressure calculation portion 108 performs a blood pressure calculation process by a volume vibration method (step S26). The blood pressure calculation process herein is not particularly limited. By applying a predetermined algorithm to the arterial volume signal and the cuff pressure, the maximum and minimum blood pressures are calculated.

Until blood pressure (maximum and minimum blood pressures) is determined (calculated) (step S28, NO), steps S22 to S26 are repeated. If the blood pressure is determined in step S26 (step S28, YES), the valve 52 is completely opened, whereby air in the airbag 21 is discharged (step S30).

The blood pressure values (the maximum and minimum blood pressures) calculated by the blood pressure calculation portion 108 are displayed on the display portion 40 and recorded in the measurement result storing region in the flash memory 43 (step S32).

In the above manner, a series of blood pressure information measurement processes end.

<Display Example and Example of Storing Measurement Data>

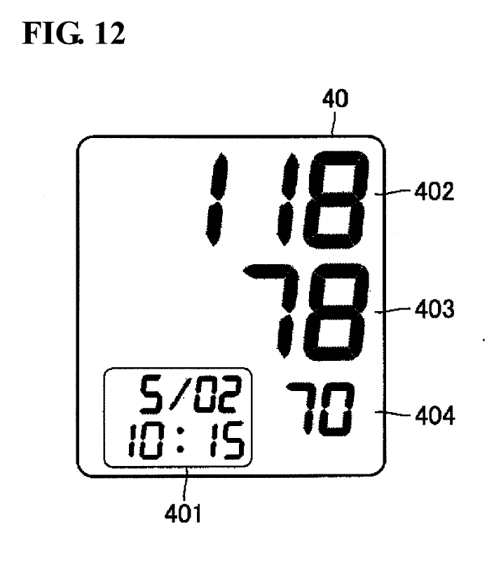

FIG. 12 is a view illustrating an example of a screen displayed in step S32 in FIG. 11.

As shown in FIG. 12, in a region 401 of the display portion 40, date and time of measurement are displayed. The date and time of measurement correspond to, for example, a point of time when the measurement switch 41B has been pressed. In regions 402 and 403 of the display portion 40, the maximum and minimum blood pressures determined in step S26 in FIG. 11 are displayed, respectively. In a region 404 of the display portion 40, a pulse rate calculated by a well known method is displayed.

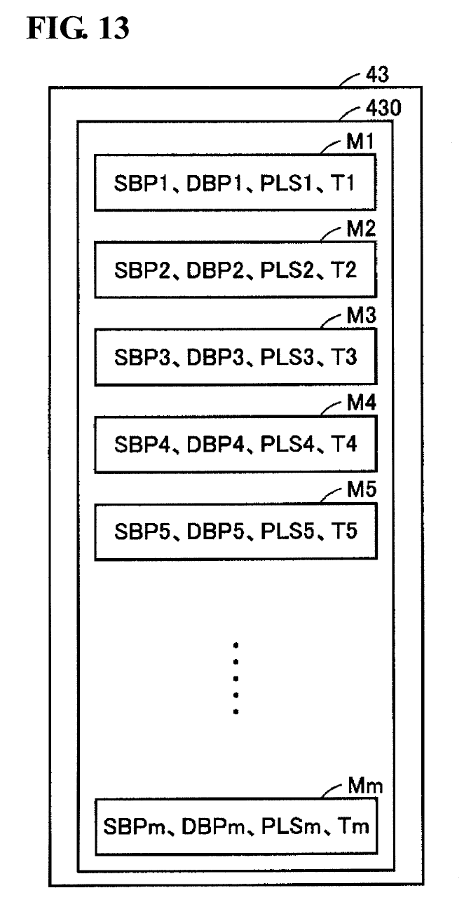

FIG. 13 is a view illustrating an example of a data structure of the measurement data.

As shown in FIG. 13, in a measurement result storing region 430, a record in which the measurement value and the date and time of measurement are associated with each other is stored as measurement data Ml to Mm (here, m=1, 2, 3, . . . ). The respective measurement data includes maximum blood pressure data SBP showing the maximum blood pressure, minimum blood pressure data DBP showing the minimum blood pressure, pulse rate data PLS showing the pulse rate, and date and time of measurement data T. The measurement value and the data and time of measurement may be stored by being associated with each other, and are not limited to the storage manner using a record.

In the embodiment, the maximum and minimum blood pressures are calculated based on the arterial volume signal detected by the sensors for measurement and the cuff pressure detected by the pressure sensor 32. However, the maximum and minimum blood pressures may not be necessarily measured as blood pressure information. For example, blood pressure (pressure pulse waves) may be serially measured by the volume compensation method, based on the arterial volume signal and the cuff pressure. Alternatively, AI may be calculated based on the waveform of the pulse wave.

As described above, according to one or more embodiments of the present invention, while the cuff pressure is gradually increased, the volume pulse waves are obtained from all combinations while the combinations of the light emitting elements 71 and the light receiving elements 72 are switched. As a result, it is possible to detect optimal sensors for detecting the volume pulse wave in the blood pressure information measurement, as the sensors for measurement. Therefore, it is possible to save the user (a subject representatively) from the bother of aligning the position of the sensor on a measurement site. Moreover, even a subject who has difficulty in finding the position of the artery can detect the optimal sensors.

According to one or more embodiments of the present invention, prior to the blood pressure information measurement, a tendency of sensor output in a case where the measurement site is compressed by the cuff 20 is obtained for each combination of sensors. As a result, even if the artery is included in the measurement region of the sensor when the cuff 20 is applied, it is possible to exclude a combination of sensors that cannot accurately detect the volume pulse wave because the position of the sensors deviates or the sensors slant due to the compression of the measurement site, from the sensors for measurement. Consequently, it is possible to prevent inaccurate detection of the volume pulse wave in the blood pressure information measurement even though the position of the sensors are aligned well.

Furthermore, by a simple configuration in which only a plurality of pairs of sensors is provided, it is possible to improve the accuracy of the blood pressure information to be measured.

In addition, both the value corresponding to maximum blood pressure and the value corresponding to minimum blood pressure, which are used for detecting the sensors for measurement, are calculated by the blood pressure estimation portion 103, in parallel with obtaining the arterial volume signal from each combination of sensors. Accordingly, an extra process time for obtaining these values is not necessary, and it is possible to obtain values close to the actual maximum and minimum blood pressures of the subject.

According to one or more embodiments of the present invention, a process for obtaining the arterial volume signal from each combination of sensors and the blood pressure estimation process are performed while the pressure is increased. However, these processes may be performed while the pressure is reduced.

Although the cuff 20 was described as being applied on the wrist, the cuff 20 may be applied on the upper arm. In this case, according to one or more embodiments of the present invention, the distance between one end and the other end of the elements lined up in the longitudinal direction (row direction) of the cuff 20 be arranged so as to cover about a third of the circumference of the upper arm of a typical male adult, for example.

### Modified Example 1

According to one or more embodiments of the present invention, in order to detect the sensors for measurement, 2 envelopes PGdia and PGsys of the arterial volume signal are used. However, an average movement value of the arterial volume signal from the measurement starting point may be used instead of the envelopes.

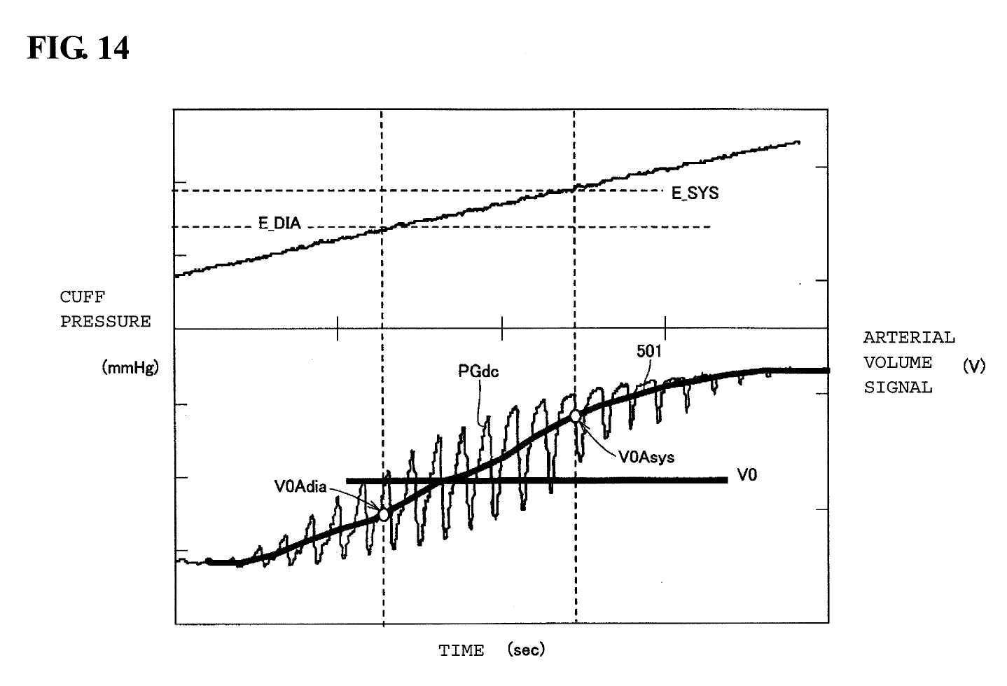

FIG. 14 is a view illustrating a line 501 connecting average movement values of the arterial volume signal PGdc from the measurement starting point. As shown in FIG. 14, a value of the line 501 obtained when the cuff pressure is the value corresponding to minimum blood pressure E_DIA is defined as a volume value V0Adia, and a value of the line 501 obtained when the value corresponding to maximum blood pressure E_SYS is defined as a volume value V0Asys, in the same manner as described above. If so, even when the average movement value is used, a combination of the light emitting element and the light receiving element in which the relationship of “V0Adia≦V0≦V0Asys” is satisfied, and a difference between the V0Adia and the V0 and a difference between the V0Asys and the V0 are within a predetermined value is determined as the sensors for measurement.

In addition, for example, when the sensors for measurement are determined based only on the relationship between the value corresponding to maximum blood pressure E_SYS and the specific volume value V0, it is possible to determine the sensors as long as the relationship of “V0≦V0Asys” is satisfied, and a difference between V0 and V0Asys is within a predetermined value.

Furthermore, the sensors for measurement may be detected by using the average of the arterial volume signal for each pulse instead of the 2 envelopes PGdia and PGsys.

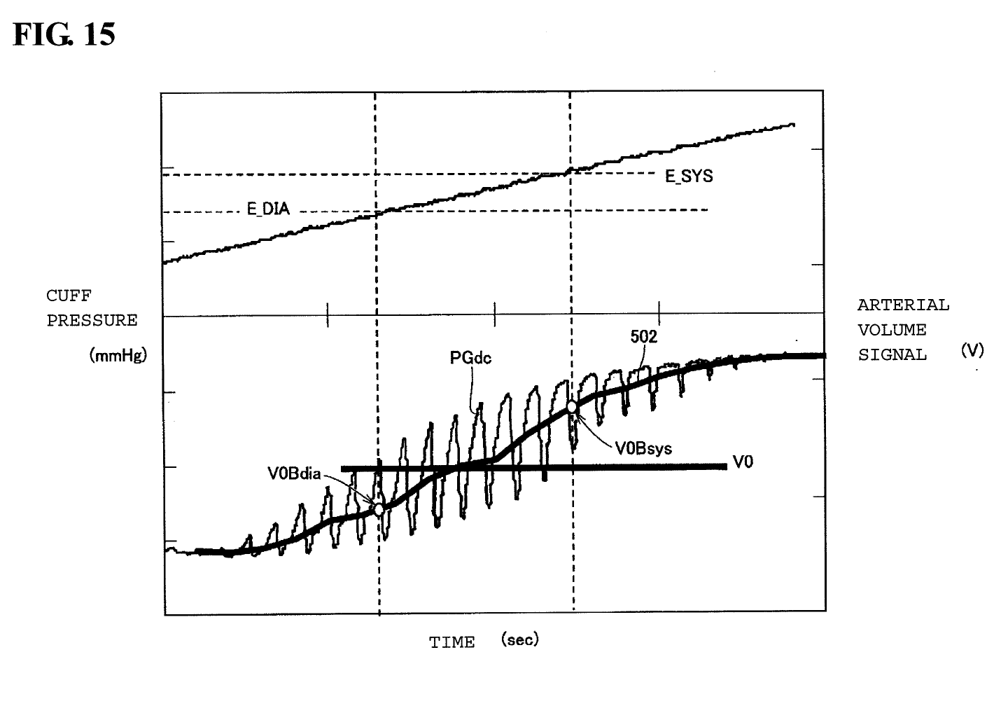

FIG. 15 is a view illustrating a line 502 connecting averages of the arterial volume signal PGdc for each pulse. As shown in FIG. 15, in this case, similarly, a value of the line 502 obtained when the cuff pressure is the value corresponding to minimum blood pressure E_DIA is defined as a volume value V0Bdia, and a value of the line 502 obtained when the cuff pressure is the value corresponding to maximum blood pressure E_SYS is defined as a volume value V0Bsys. If so, a combination of the light emitting element and the light receiving element in which the relationship of “V0Bdia≦V0≦V0Bsys” is satisfied, and a difference between the V0Bdia and the V0 and a difference between the V0Bsys and the V0 are within a predetermined value is determined as the sensors for measurement.

In this case, similarly, when the sensors for measurement are determined based only on the relationship between the value corresponding to maximum blood pressure E_SYS and the specific volume value V0, for example, it is possible to determine the sensors as long as the relationship of “V0≦V0Bsys” is satisfied, and a difference between V0 and V0Bsys is within a predetermined value.

### Modified Example 2

According to one or more embodiments of the present invention, as the arterial volume sensor, the photoelectric sensor 70 is used. However, instead of the photoelectric sensor 70, an impedance sensor including a plurality of pairs of electrodes for applying current (hereinafter, referred to as “current electrodes”) and electrodes for measuring voltage (voltage electrodes) may be used.

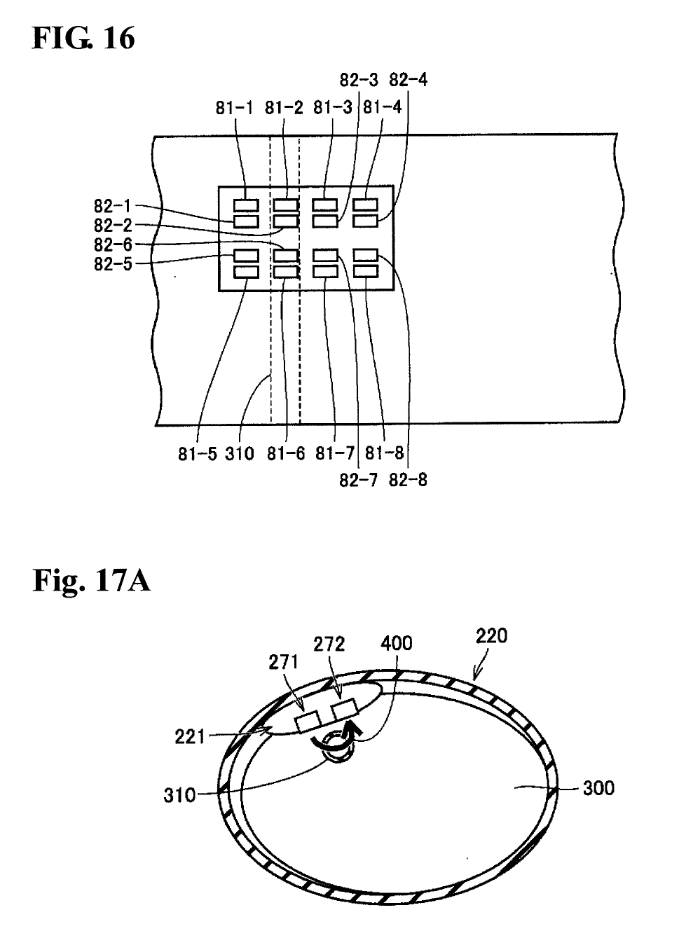

FIG. 16 is a view illustrating an arrangement example of a plurality of pairs of current electrodes 81-1 to 81-8 and voltage electrodes 82-1 to 82-8.

In order to detect the volume pulse wave by the impedance sensor, the detection process portion 104 causes the switching portion 75 to sequentially select a pair of the current electrodes 81, and causes the switching portion 76 to sequentially select a pair of the voltage electrodes 82.

Instead of the light emitting element driving circuit 73, a constant current generating portion for applying current to a pair of electrodes among the current electrodes 81-1 to 81-8 may be provided. In this case, the arterial volume detection circuit 74 may measure impedance based on voltage detected by a pair of electrodes among the voltage electrodes 82-1 to 82-8, and detect the arterial volume based on the measured impedance.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 blood pressure information measurement device (sphygmomanometer), 10 body portion, 20,220 cuff, 21,221 airbag, 30 air system, 31 air tube, 32 pressure sensor, 33 oscillation circuit, 40 display portion, 41 operation portion, 42 memory portion, 43 flash memory, 44 power supply, 45 clocking portion, 46 interface portion, 51 pump, 52 valve, 53 pump driving circuit, 54 valve driving circuit, 70 photoelectric sensor, 71-1,71-2, 71-3,71-4, 71-5,71-6, 71-7,71-8,271 light emitting elements, 72-1,72-2, 72-3,72-4, 72-5,72-6, 72-7,72-8,272 light receiving elements, 73 light emitting element driving circuit, 74 arterial volume detection circuit, 75,76 switching portions, 81 current electrode, 82 voltage electrode, 100 CPU, 102 pressure increase control portion, 103 blood pressure estimation portion, 104 detection process portion, 106 pressure reduction control portion, 108 blood pressure calculation portion, 132 recording medium, 300 body, 310 artery, 400 optical axis, 410 substrate, 430 measurement result storing region.

1. A blood pressure information measurement device for measuring blood pressure information by detecting an arterial volume, comprising:
a cuff to be wound around a predetermined measurement site;
adjustment portions that adjust internal pressure of the cuff by increasing or reducing the pressure;
pressure detection portions that detect a cuff pressure showing the internal pressure of the cuff; and
a volume detection portion that is disposed in a predetermined position of the cuff and that detects arterial volume signals showing the arterial volume,
wherein the volume detection portion comprises a plurality of pairs of first and second sensors, further including comprising:
driving control portions for that gradually increase or reduce the cuff pressure until the cuff pressure reaches a specific pressure value by controlling driving of the adjustment portions; and
detection process portions that perform a process that detects a pair of sensors for measurement among the plurality of pairs of the first and second sensors,
wherein the process performed by the detection process portions includes comprises:
a step of obtaining volume pulse waves by obtaining arterial volume signals from the volume detection portions for each combination of the plurality of pairs of the first and second sensors in parallel with the control performed by the driving control portion:
a step of extracting specific volume values in points where an amount of volume change becomes maximal for each volume pulse wave corresponding to the combinations when the cuff pressure has reached the specific pressure value by the driving control portions; and
a step of determining a combination of the first and second sensors in which at least one of a relationship between the specific volume value and a first volume value at a point of time when the cuff pressure becomes a value corresponding to a maximum arterial blood pressure satisfies a predetermined first relationship, and a relationship between the specific volume value and a second volume value at a point of time when the cuff pressure becomes a value corresponding to a minimum arterial blood pressure satisfies a predetermined second relationship, as sensors for measurement, and
wherein the blood pressure information measurement device further comprises a blood pressure information calculation portion that measures the blood pressure information by using the sensors for measurement determined in the step of determining the sensors for measurement.

2. The blood pressure information measurement device according to claim 1,
wherein the predetermined first relationship shows a relationship in which:
the specific volume value is equal to or smaller than the first volume value; and
a difference between the specific volume value and the first volume value is within a predetermined value.

3. The blood pressure information measuring measurement device according to claim 1,
wherein the predetermined second relationship shows a relationship in which:
the specific volume value is equal to or larger than the second volume value; and
a difference between the specific volume value and the second volume value is within a predetermined value.

4. The blood pressure information measurement device according to claim 1, further comprising a switching portion that switches the combinations of the first and second sensors,
wherein in the step of obtaining the volume pulse waves, the volume pulse wave for each of the combinations is obtained while the switching portion switches the combinations of the first and the second sensors.

5. The blood pressure information measurement device according to claim 4,
wherein in the step of obtaining the volume pulse waves, the combinations of the first and the second sensors are sequentially switched at a same cuff pressure.

6. The blood pressure information measurement device according to claim 1, further comprising a storage portion that stores values of the arterial volume signals obtained in the step of obtaining the volume pulse waves by associating the values with the cuff pressure for each of the combinations,
wherein in the step of determining the sensors for measurement, a first envelope tangent to points where the arterial volume becomes maximal for each of pulse wave components included in the volume pulse wave and a second envelope connecting points where the arterial volume becomes minimal are extracted for each volume pulse wave, a value of the first envelope that corresponds to a point of time when the cuff pressure of a value corresponding to the maximum blood pressure is detected is determined as a first volume value, and a value of the second envelope that corresponds to a point of time when the cuff pressure of a value corresponding to the minimum blood pressure is detected is determined as a second volume value.

7. The blood pressure information measurement device according to claim 1, further comprising a storage portion that stores the values of the arterial volume signals obtained in the step of obtaining the volume pulse waves by associating the value with the cuff pressure for each of the combinations,
wherein the value of the arterial volume signal increases according to the increase in the cuff pressure, and
wherein in the step of determining the sensors for measurement, average movement values of the arterial volume signals from the detection starting point are calculated, an average movement value that corresponds to a point of time when the cuff pressure of a value corresponding to the maximum blood pressure is detected is determined as the first volume value, and an average movement value that corresponds to a point of time when the cuff pressure of a value corresponding to the minimum blood pressure is detected is determined as the second volume value.

8. The blood pressure information measurement device according to claim 1, further comprising a storage portion that stores the values of the arterial volume signals obtained in the step of obtaining the volume pulse waves by associating the values with the cuff pressure for each of the combinations,
wherein the value of the arterial volume signal increases according to the increase in the cuff pressure, and
wherein in the step of determining the sensors for measurement, an average of the volume pulse wave for one pulse that corresponds to a point of time when the cuff pressure of a value corresponding to the maximum blood pressure is detected is determined as the first volume value, and an average of the volume pulse wave for one pulse that corresponds to a point of time when the cuff pressure of a value corresponding to the minimum blood pressure is detected is determined as the second volume value.

9. The blood pressure information measurement device according to claim 1, further comprising a blood pressure estimation portion that estimates a maximum arterial blood pressure value and a minimum arterial blood pressure value in parallel with the control performed by the driving control portions,
wherein each of the value corresponding to the maximum blood pressure and the value corresponding to the minimum blood pressure indicate the maximum blood pressure value and the minimum blood pressure value that are estimated by the blood pressure estimation portion.

10. The blood pressure information measurement device according to claim 1,
wherein the volume detection portion is a photoelectric sensor, and
wherein the first and second sensors are a light emitting element and a light receiving element, respectively.

11. The blood pressure information measuring measurement device according to claim 1,
wherein the volume detection portion is an impedance sensor, and
wherein the first and second sensors are a current applying electrode and a voltage measuring electrode, respectively.

12. A method of measuring blood pressure information by detecting an arterial volume by using a blood pressure information measuring measurement device comprising a cuff for being wound around a predetermined measurement site and a plurality of pairs of first and second sensors in a predetermined position of the cuff, the method comprising:
gradually increasing or reducing cuff pressure until the cuff pressure reaches a specific pressure value;
obtaining volume pulse waves by obtaining arterial volume signals showing an arterial volume from the sensor pairs for each of the combinations of the plurality of pairs of the first and second sensors in parallel with the increase or reduction of the cuff pressure;
extracting a specific volume value at a point where an amount of volume change becomes maximal for each volume pulse wave corresponding to the combination, when the cuff pressure has reached the specific pressure value;
determining a combination of the first and second sensors in which at least one of a relationship between the specific volume value and a first volume value at a point of time when the cuff pressure becomes a value corresponding to a maximum arterial blood pressure satisfies a predetermined first relationship, and a relationship between the specific volume value and a second volume value at a point of time when the cuff pressure becomes a value corresponding to a minimum arterial blood pressure satisfies a predetermined second relationship, as sensors for measurement; and
measuring blood pressure information by using the determined sensors for measurement.
